(19) United States
(12) Patent Application Publication
(10) Pub. No.: US 2009/0041675 A1
(43) Pub. Date: Feb. 12, 2009

(54) Organic compounds

(76) Inventor: Alexandre Trifilieff, Dietwiller (FR)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/921,189

(22) PCT Filed: May 30, 2006
(86) PCT No.: PCT/EP2006/005154

§ 371 Date: Nov. 28, 2007

(30) Foreign Application Priority Data
May 31, 2005 (GB) 0511066.3

(51) Int. Cl. A61K 9/72; C07D 215/12; A61K 31/47; A61K 31/519; A61P 11/00; C07D 215/227; A61K 31/4985; A61K 31/53

(52) U.S. Cl. 424/40; 546/176; 514/311; 514/262.1; 514/312; 546/157; 514/250; 514/243

(57) ABSTRACT

A medicament comprising, separately or together, (A) a compound of formula I

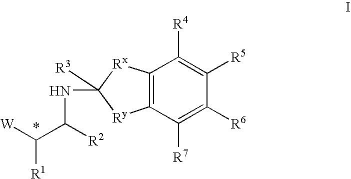

in free or salt or solvate form, where W, R^{x}, R^{y}, R¹, R², R³, R⁴, R⁵, R⁶ and R⁷ have the meanings as indicated in the specification, and (B) one or more of compounds selected from the group consisting of PDE4 inhibitors and PDE5 inhibitors, for simultaneous, sequential or separate administration in the treatment of an inflammatory or obstructive airways disease.

This invention relates to organic compounds and their use as pharmaceuticals, in particular for the treatment of inflammatory or obstructive airways diseases.

In one aspect, the present invention provides a medicament comprising, separately or together,
(A) a compound of formula I

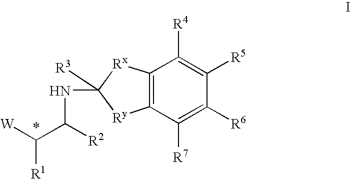

in free or salt or solvate form, where
W is a group of formula

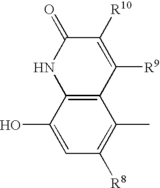

R^{x} and R^{y} are both —CH₂— or —(CH₂)₂—;
R¹ is hydrogen, hydroxy, or C₁-C₁₀-alkoxy;
R² and R³ are each independently hydrogen or C₁-C₁₀-alkyl;
R⁴, R⁵, R⁶ and R⁷ are each independently hydrogen, halogen, cyano, hydroxy, C₁-C₁₀-alkoxy, C₆-C₁₀-aryl, C₁-C₁₀-alkyl, C₁-C₁₀-alkyl substituted by one or more halogen atoms or one or more hydroxy or C₁-C₁₀-alkoxy groups, C₁-C₁₀-alkyl interrupted by one or more hetero atoms, C₂-C₁₀-alkenyl, trialkylsilyl, carboxy, C₁-C₁₀-alkoxycarbonyl, or —CONR¹¹R¹² where R¹¹ and R¹² are each independently hydrogen or C₁-C₁₀-alkyl,
or R⁴ and R⁵, R⁵ and R⁶, or R⁶ and R⁷ together with the carbon atoms to which they are attached denote a 5-, 6- or 7-membered carbocyclic ring or a 4- to 10-membered heterocyclic ring; and
R⁸, R⁹ and R¹⁰ are each independently hydrogen or C₁-C₄-alkyl; and
(B) one or more of compounds selected from the group consisting of PDE4 inhibitors and PDE5 inhibitors;
   for simultaneous, sequential or separate administration in the treatment of an inflammatory or obstructive airways disease.

In another aspect, the present invention provides a method of treating an inflammatory or obstructive airways disease which comprises administering to a subject in need of such treatment effective amounts of (A) as hereinbefore defined and (B) as hereinbefore defined.

In a further aspect, the present invention provides a pharmaceutical composition comprising a mixture of effective amounts of (A) as hereinbefore defined and (B) as hereinbefore defined, optionally together with at least one pharmaceutically acceptable carrier.

The invention further provides the use of (A) as hereinbefore defined and (B) as hereinbefore defined in the preparation of a medicament for combination therapy by simultaneous, sequential or separate administration of (A) and (B) in the treatment of an inflammatory or obstructive airways disease.

Terms used in the specification have the following meanings:

“Optionally substituted” as used herein means the group referred to can be substituted at one or more positions by any one or any combination of the radicals listed thereafter.

“Halo” or “halogen” as used herein denotes a element belonging to group 17 (formerly group VII) of the Periodic Table of Elements, which may be, for example, fluorine, chlorine, bromine or iodine. Preferably halo or halogen is fluorine or chlorine.

“C₁-C₁₀-alkyl” as used herein denotes straight chain or branched alkyl that contains one to ten carbon atoms. Preferably, C₁-C₁₀-alkyl is C₁-C₄-alkyl.

“C₁-C₁₀-alkylene” as used herein denotes a straight chain or branched alkylene that contains one to ten carbon atoms. Preferably C₁-C₁₀-alkylene is C₁-C₄ alkylene, especially ethylene or methylethylene.

“C₂-C₁₀-alkenyl” as used herein denotes straight chain or branched hydrocarbon chains that contain two to ten carbon atoms and one or more carbon-carbon double bonds. Preferably “C₂-C₁₀-alkenyl” is “C₂-C₄-alkenyl”.

“C₂-C₁₀-alkynyl” as used herein denotes straight chain or branched hydrocarbon chains that contain two to ten carbon atoms and one or more carbon-carbon triple bonds. Preferably “C₂-C₁₀-alkynyl” is “C₂-C₄-alkynyl”.

“5-, 6 or 7-membered carbocyclic ring” as used herein denotes a carbocyclic group having 5 to 7 ring carbon atoms, either cycloaliphatic, such as a C₅-C₇-cycloalkyl, or aromatic, such as phenyl, which can be substituted by one or more, usually one or two, C₁-C₄-alkyl groups.

“C₃-C₁₀-cycloalkyl” as used herein denotes cycloalkyl having 3 to 10 ring carbon atoms, for example a monocyclic group such as a cyclopropyl, cyclobutyl, cyclopentyl, cyclohexyl, cycloheptyl, cyclooctyl, cyclononyl or cyclodecyl, any of which can be substituted by one or more, usually one or two, C₁-C₄-alkyl groups, or a bicyclic group such as bicycloheptyl or bicyclooctyl. Preferably C₃-C₁₀-cycloalkyl is C₃-C₆-cycloalkyl, for example cyclopropyl, cyclobutyl, cyclopentyl, cyclohexyl or cycloheptyl.

“C₁-C₁₀-haloalkyl” as used herein denotes C₁-C₁₀-alkyl as hereinbefore defined substituted by one or more halogen atoms, preferably one, two or three halogen atoms.

“C₁-C₁₀-alkylamino” and “di(C₁-C₁₀-alkyl)amino” as used herein denote amino substituted respectively by one or two C₁-C₁₀-alkyl groups as hereinbefore defined, which may be the same or different. Preferably C₁-C₁₀-alkylamino and di(C₁-C₁₀-alkyl)amino are respectively C₁-C₄-alkylamino and di(C₁-C₄-alkyl)amino.

“C₁-C₁₀-alkylthio” as used herein denotes straight chain or branched alkylthio having 1 to 10 carbon atoms. Preferably, C₁-C₁₀-alkylthio is C₁-C₄-alkylthio.

“C₁-C₁₀-alkoxy” as used herein denotes straight chain or branched alkoxy that contains 1 to 10 carbon atoms. Preferably, C₁-C₁₀-alkoxy is C₁-C₄-alkoxy.

“C₁-C₁₀-alkoxy-C₁-C₁₀-alkyl” as used herein denotes C₁-C₁₀-alkyl as hereinbefore defined substituted by C₁-C₁₀-alkoxy. Preferably, C₁-C₁₀-alkoxy-C₁-C₁₀-alkyl is C₁-C₄-alkoxy-C₁-C₄-alkyl.

“C₁-C₁₀-alkoxycarbonyl” as used herein denotes C₁-C₁₀-alkoxy as hereinbefore defined linked through an oxygen atom thereof to a carbonyl group.

“C₆-C₁₀-aryl” as used herein denotes a monovalent carbocyclic aromatic group that contains 6 to 10 carbon atoms and which may be, for example, a monocyclic group such as phenyl or a bicyclic group such as naphthyl. Preferably C₆-C₁₀-aryl is C₆-C₈-aryl, especially phenyl.

“C₆-C₁₀-arylsulfonyl” as used herein denotes C₆-C₁₀-aryl as hereinbefore defined linked through a carbon atom thereof to a sulfonyl group. Preferably C₆-C₁₀-arylsulfonyl is C₆-C₈-arylsulfonyl.

“C₇-C₁₄-aralkyl” as used herein denotes alkyl, for example C₁-C₄-alkyl as hereinbefore defined, substituted by aryl, for example C₆-C₁₀-aryl as hereinbefore defined. Preferably, C₇-C₁₄-aralkyl is C₇-C₁₀-aralkyl such as phenyl-C₁-C₄-alkyl, particularly benzyl or 2-phenylethyl.

“C₇-C₁₄-aralkyloxy” as used herein denotes alkoxy, for example C₁-C₄-alkoxy as hereinbefore defined, substituted by aryl, for example C₆-C₁₀-aryl. Preferably, C₇-C₁₄-aralkyloxy is C₇-C₁₀-aralkyloxy such as phenyl-C₁-C₄-alkoxy, particularly benzyloxy or 2-phenylethoxy.

Ar as used herein may be, for example, phenylene which is unsubstituted or substituted by one or more substituents selected from halogen, hydroxy, C₁-C₁₀-alkyl, C₁-C₁₀-alkoxy, C₁-C₁₀-alkoxy-C₁-C₁₀-alkyl, phenyl, or C₁-C₁₀-alkyl substituted by phenyl, C₁-C₁₀-alkoxy substituted by phenyl, C₁-C₁₀-alkyl-substituted phenyl and C₁-C₁₀-alkoxy-substituted phenyl. Preferably Ar is phenylene which is unsubstituted or substituted by one or two substituents selected from halogen, C₁-C₄-alkyl, C₁-C₄-alkoxy, or C₁-C₄-alkoxy substituted by phenyl. Preferably one substituent in Ar is para to R¹ and optional second and third substituents in Ar are meta to R¹.

“4- to 10-membered heterocyclic ring having at least one ring nitrogen, oxygen or sulphur atom” as used herein may be, for example, pyrrole, pyrrolidine, pyrazole, imidazole, triazole, tetrazole, thiadiazole, oxazole, isoxazole, thiophene, thiazole, isothiazole, oxadiazole, pyridine, pyrazine, pyridazine, pyrimidine, piperidine, piperazine, triazine, oxazine, morpholino, quinoline, isoquinoline, naphthyridine, indane or indene. Preferred heterocyclic rings include thiazole, pyrrolidine, piperidine, azacycloheptane and isoxazole.

“4 to 10-membered heterocyclyl-C₁-C₁₀-alkyl” denotes alkyl, for example C₁-C₁₀-alkyl as hereinbefore defined, substituted by a 4- to 10-membered heterocyclic ring as hereinbefore defined. Preferably, 4- to 10-membered heterocyclyl-C₁-C₁₀-alkyl is C₁-C₄-alkyl substituted by a 4- to 8-membered heterocyclic ring having at least one ring nitrogen, oxygen or sulphur atom.

“C₁-C₄-alkylsulfonyl” denotes sulfonyl substituted by C₁-C₄-alkyl as hereinbefore defined. “Hydroxy-C₁-C₄-alkyl” denotes C₁-C₄-alkyl as hereinbefore defined substituted by one or more, preferably one, two or three hydroxy groups.

R¹³ and R¹⁴ together with the carbon atoms to which they are attached as a cycloaliphatic ring may be, for example, a cyclopentane ring, optionally substituted by one or two C₁-C₄-alkyl groups, a cyclohexane ring, optionally substituted by one or two C₁-C₄-alkyl groups, or a cycloheptane ring, preferably a cyclopentane ring.

Throughout this specification and in the claims that follow, unless the context requires otherwise, the word “comprise”, or variations such as “comprises” or “comprising”, should be understood to imply the inclusion of a stated integer or step or group of integers or steps but not the exclusion of any other integer or step or group of integers or steps.

Preferred compounds of formula I include those wherein

R⁸, R⁹ and R¹⁰ are each H, R¹ is OH, R² and R³ are each H and
(i) R^{x} and R^{y} are both —CH₂—, and R⁴ and R⁷ are each CH₃O— and R⁵ and R⁶ are each H;
(ii) R^{x} and R^{y} are both —CH₂—, and R⁴ and R⁷ are each H and R⁵ and R⁶ are each CH₃CH₂—;
(iii) R^{x} and R^{y} are both —CH₂—, and R⁴ and R⁷ are each H and R⁵ and R⁶ are each CH₃—;
(iv) R^{x} and R^{y} are both —CH₂—, and R⁴ and R⁷ are each CH₃CH₂— and R⁵ and R⁶ are each H;
(v) R^{x} and R^{y} are both —CH₂—, and R⁴ and R⁷ are each H and R⁵ and R⁶ together denote —(CH₂)₄—;
(vi) R^{x} and R^{y} are both —CH₂—, and R⁴ and R⁷ are each H and R⁵ and R⁶ together denote —O(CH₂)₂O—;
(vii) R^{x} and R^{y} are both —CH₂—, and R⁴ and R⁷ are each H and R⁵ and R⁶ are each CH₃(CH₂)₃—;
(viii) R^{x} and R^{y} are both —CH₂—, and R⁴ and R⁷ are each H and R⁵ and R⁶ are each CH₃(CH₂)₂—;
(ix) R^{x} and R^{y} are both —(CH₂)₂—, R⁴, R⁵, R⁶ and R⁷ are each H; or
(x) R^{x} and R^{y} are both —CH₂—, and R⁴ and R⁷ are each H and R⁵ and R⁶ are each CH₃OCH₂—.

These include 8-hydroxy-5-[1-hydroxy-2-(indan-2-ylamino)-ethyl]-1H-quinolin-2-one, 5-[2-(5,6-dimethoxy-indan-2-ylamino)-1-hydroxy-ethyl]-8-hydroxy-1H-quinolin-2-one, 5-[2-(5,6-diethyl-indan-2-ylamino)-1-hydroxy-ethyl]-8-hydroxy-3-methyl-1H-quinolin-2-one, 5-[2-(5,6-diethyl-indan-2-ylamino)-1-hydroxy-ethyl]-8-methoxy-methoxy-6-methyl-1H-quinolin-2-one, 5-[2-(5,6-diethyl-indan-2-ylamino)-1-hydroxy-ethyl]-8-hydroxy-6-methyl-1H-quinolin-2-one, 8-hydroxy-5-[2-(5,6-diethyl-indan-2-ylamino)-1-hydroxy-ethyl]-3,4-dihydro-1H-quinolin-2-one, 5-[(R)-2-(5,6-diethyl-2-methyl-indan-2-ylamino)-1-hydroxy-ethyl]-8-hydroxy-1H-quinolin-2-one, (S)-5-[2-(4,7-diethyl-indan-2-ylamino)-1-hydroxy-ethyl]-8-hydroxy-1H-quinolin-2-one hydrochloride, 5-[(R)-1-hydroxy-2-(6,7,8,9-tetrahydro-5H-benzocyclohepten-7-ylamino)-ethyl]-8-hydroxy-1H-quinolin-2-one hydrochloride, (R)-5-[2-(5,6-diethyl-indan-2-ylamino)-1-hydroxy-ethyl]-8-hydroxy-1H-quinolin-2-one maleate, (R)-5-[2-(5,6-diethyl-indan-2-ylamino)-1-hydroxy-ethyl]-8-hydroxy-1H-quinolin-2-one hydrochloride, (R)-8-hydroxy-5-[(S)-1-hydroxy-2-(4,5,6,7-tetramethyl-indan-2-ylamino)-ethyl]-1H-quinolin-2-one, 8-hydroxy-5-[(R)-1-hydroxy-2-(2-methyl-indan-2-ylamino)-ethyl]-1H-quinolin-2-one, 5-[2-(5,6-diethyl-indan-2-ylamino)-ethyl]-8-hydroxy-1H-quinolin-2-one, 8-hydroxy-5-[(R)-1-hydroxy-2-(2-methyl-2,3,5,6,7,8-hexahydro-1H-cyclopenta[b]naphthalen-2-ylamino)-ethyl]-1H-quinolin-2-one, and 5-[(S)-2-(2,3,5,6,7,8-hexahydro-1H-cyclopenta[b]naph-thalen-2-ylamino)-1-hydroxy-ethyl]-8-hydroxy-1H-quinolin-2-one.

An especially preferred compound of formula I is a compound of formula II

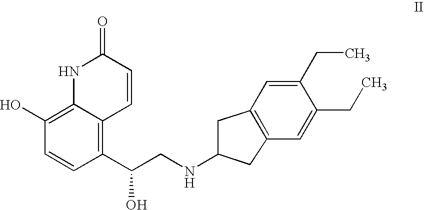

in free or pharmaceutically acceptable salt or solvate form, especially the maleate salt, namely (R)-5-[2-(5,6-diethyl-indan-2-ylamino)-1-hydroxyethyl]-8-hydroxy-1H-quinolin-2-one maleate.

Compounds of formula I in free or salt or solvate form may be prepared by using the procedures described in international patent application WO 2000/075114, the contents of which is incorporated herein by reference.

The compound of formula II may be prepared in free or salt or solvate form by reacting (R)-8-benzyloxy-5-oxiranylcarbostyril with 5,6-diethylindan-2-ylamine to give 8-benzyloxy-5-[(R)-2-(5,6-diethyl-indan-2-ylamino)-1-hydroxy-ethyl]-1H-quinolin-2-one, subjecting the latter to a deprotecting reaction to replace the benzyl group by hydrogen, and recovering the resultant compound of formula II in free or salt or solvate form. Such a process ins described in WO 2004/76422, the contents of which is incorporated herein by reference. (R)-8-benzyloxy-5-oxiranylcarbostyril may be prepared as described in WO 1995/25104. 5,6-Diethyl-indan-2-ylamine may be prepared as described in WO 2003/76387.

Compounds of formula I in free form may be converted into salt form, and vice versa, in a conventional manner. The compounds in free or salt form can be obtained in the form of hydrates or solvates containing a solvent used for crystallisation. Compounds of formula I can be recovered from reaction mixtures and purified in a conventional manner. Isomers, such as enantiomers, may be obtained in a conventional manner, e.g. by fractional crystallisation or asymmetric synthesis from correspondingly asymmetrically substituted, e.g. optically active, starting materials.

Pharmaceutically acceptable salts of the compound of formula I may be acid addition salts, including those of inorganic acids, for example hydrohalic acids such as hydrofluoric acid, hydrochloric acid, hydrobromic acid or hydroiodic acid, nitric acid, sulfuric acid, phosphoric acid; and organic acids such as formic acid, acetic acid, propionic acid, butyric acid, benzoic acid, o-hydroxybenzoic acid, p-hydroxybenzoic acid, p-chlorobenzoic acid, diphenylacetic acid, triphenylacetic acid, 1-hydroxynaphthalene-2-carboxylic acid, 3-hydroxynaphthalene-2-carboxylic acid, aliphatic hydroxy acids such as lactic acid, citric acid, tartaric acid or malic acid, dicarboxylic acids such as fumaric acid, maleic acid or succinic acid, and sulfonic acids such as methanesulfonic acid or benzenesulfonic acid. These salts may be prepared from compounds of formula I by known salt-forming procedures. Pharmaceutically acceptable solvates are generally hydrates.

A PDE4 inhibitor is a substance or agent that exhibits cyclic nucleotide phosphodiesterase (PDE) isoenzyme inhibiting activity, selective for type 4 isoenzyme. Such substances possess anti-inflammatory, anti-airways hyperreactivity and bronchodilator properties. They can also possess immunosuppressive and TNFα secretion inhibitory activities. PDE4 inhibition activity may be measured using the PDE4 isoenzyme inhibition assay described in WO 03/39544

Suitable PDE4 inhibitors include cilomilast (Ariflo® GlaxoSmithKline), roflumilast (Byk Gulden), V-11294A (Napp), BAY19-8004 (Bayer), SCH-351591 (Schering-Plough), Arofylline (Almirall Prodesfarma), PD189659/PD168787 (Parke-Davis), AWD-12-281 (Asta Medica), CDC-801 (Celgene), SelCID™ CC-10004 (Celgene), VM554/UM565 (Vernalis), T-440 (Tanabe), KW-4490 (Kyowa Hakko Kogyo), and those disclosed in WO 92/19594, WO 93/19749, WO 93/19750, WO 93/19751, WO 98/18796, WO 99/16766, WO 01/13953, WO 03/104204, WO 03/104205, WO 03/39544, WO 04/000814, WO 04/000839, WO 04/005258, WO 04/018450, WO 04/018451, WO 04/018457, WO 04/018465, WO 04/018431, WO 04/018449, WO 04/018450, WO 04/018451, WO 04/018457, WO 04/018465, WO 04/019944, WO 04/019944, WO 04/019945, WO 04/045607, WO 04/037805, WO 04/063197, WO 04/103998, WO 04/111044, WO 05/012252, WO 05/012253 and WO 05/013995, WO 05/030212, WO 05/030725, WO 05/087744, WO 05/087745, WO 05/087749 and WO 05/090345.

A PDE5 inhibitor is a substance or agent that exhibits cyclic nucleotide phosphodiesterase (PDE) isoenzyme inhibiting activity, selective for type 5 isoenzyme. Such substances are useful in the treatment of sexual dysfunction, including male erectile dysfunction and female sexual dysfunction, premature labour, dysmenorrhoea, benign prostatic hyperplasia, bladder outlet obstruction, incontinence, stable, unstable and variant (Prinzmetal) angina, hypertension, pulmonary hypertension, congestive heart failure, atherosclerosis, conditions of reduced blood vessel patency, e.g. post-percutaneous transluminal coronary angioplasty, peripheral vascular disease, bronchitis, asthma, allergic rhinitis, glaucoma, tinnitus, diseases characterised by disorders of gut motility, e.g. irritable bowel syndrome, pre-eclampsia, Kawasaki's syndrome, nitrate tolerance, multiple sclerosis, peripheral diabetic neuropathy, stroke, Alzheimer's disease, acute respiratory failure, psoriasis, skin necrosis, cancer, metastasis, baldness, nutcracker oesophagus, anal fissure and hypoxic vasoconstriction. PDE5 inhibition activity may be measured using the PDE5 isoenzyme inhibition assay described in WO 2001/77110.

Suitable PDE5 inhibitors include pyrazolopyrimidinones disclosed in EP 463756, EP 526004, WO 94/28902, WO 96/16657 or WO 98/49166; 5-substituted pyrazole[4,3-d]pyrimidin-7-ones disclosed in EP 201188; griseolic acid derivatives disclosed in EP 214708 and EP 319050; 2-phenylpurinone derivatives disclosed in EP 293063; phenylpyridone derivatives disclosed in EP 347027; fused pyrimidine derivatives disclosed in EP 347146; condensed pyrimidine derivatives disclosed in EP 349239; pyrimidopyrimidine derivatives disclosed in EP 351058; purine compounds disclosed in EP 352960; quinazolinone derivatives disclosed in EP 371731; phenylpyrimidone derivatives disclosed in EP 395328; imidazoquin-oxalinone derivatives or their aza analogues disclosed in EP 400583; phenylpyrimidone derivatives disclosed in EP 400799; phenylpyridone derivatives disclosed in EP 428268; pyrimidopyrimidine derivatives disclosed in EP 442204; 4-aminoquinazoline derivatives disclosed in EP 579496; 4,5-dihydro-4-oxo-pyrrolo[1,2-a]quinoxaline derivatives or their aza analogues disclosed in EP 584487; polycyclic guanine derivatives disclosed in WO91/19717; nitrogenous heterocyclic compounds disclosed in WO93/07124; 2-benzyl-polycyclic guanine derivatives disclosed in WO94/19351; quinazoline derivatives disclosed in U.S. Pat. No. 4,060,615; 6-heterocyclyl pyrazolo[3,4-d]pyrimidin-4-ones disclosed in U.S. Pat. No. 5,294,612; benzimidazoles disclosed in Japanese Kokai 5-222000; cycloheptimidazoles disclosed in European Journal of Pharmacology, vol. 251, (1994), 1; N-containing heterocycles disclosed in WO 94/22855; pyrazolopyrimidine derivatives disclosed in EP 636626; 4-aminopyrimidine derivatives disclosed in EP 640599; imidazoquinazoline derivatives disclosed in EP 668280; anthranilic acid derivatives disclosed in EP 0686625; 4-aminoquinazoline derivatives disclosed in U.S. Pat. No. 5,436,233; tetracyclic derivatives disclosed in WO 95/19978 (including tadafil); quinazoline compounds disclosed in EP 0669324; fused pyridazine compounds disclosed in EP 722936; imidazoquinoline compounds disclosed in EP 0758653; substituted pyrazoloquinolinamines disclosed in WO 96/28159; substituted pyrazolopyrimidinones disclosed in WO 96/28429; indole derivatives disclosed in WO 96/32379; benzimidazole derivatives disclosed in WO 97/03070; 2-phenyl substituted imidazotriazinone derivatives disclosed in WO 99/24433 (including vardenafil); as well as those described in WO 2001/77110.

Especially preferred PDE5 inhibitors include 5-[2-ethoxy-5-(4-methylpiperazinylsulfonyl)-phenyl]-1-methyl-3-n-propyl-1,6-dihydro-7H-pyrazolo[4,3-d]pyrimidin-7-one (sildenafil, VIAGRA®, disclosed in WO 94/28902), (6R,12aR)-2,3,6,7,12,12a-Hexahydro-2-methyl-6-(3,4-methylenedioxyphenyl)-pyrazino(2′,1′:6,1)pyrido (3,4-b)indole-1,4-dione (tadafil, CIALIS®, disclosed in WO 95/19978)), 4-ethoxy-N-propyl-N-(2-hydroxyethyl)-3-(5-methyl-4-oxo-7-propyl-3,4-dihydroimidazo[5,1-f][1,2,4]triazin-2-yl)benzenesulfonamide (vardenafil, LEVITRA®, disclosed in WO 99/24433), 4-phenyl-methylamino-6-chloro-2-(1-imidazolyl)-quinazoline (disclosed in U.S. Pat. No. 5,436,233), 4-phenylmethylamino-6-chloro-2-(3-pyridyl)quinazoline (disclosed in U.S. Pat. No. 5,436,233), 1,3-dimethyl-6-(2-propoxy-5-methanesulfonylamidophenyl)-1,5-dihydropyrazolo[3,4-d]pyrimidin-4-one (disclosed in EP 636626) and 1-cyclopentyl-3-ethyl-6-(3-ethoxy-4-pyridyl)-pyrazolo[3,4-d]pyrimidin-4-one (disclosed in U.S. Pat. No. 5,294,612).

Administration of the medicament or pharmaceutical composition as hereinbefore described, i.e. with (A) and (B) in admixture or separate, is preferably by inhalation, i.e. (A) and (B) or the mixture thereof are in inhalable form. The inhalable form of the medicament i.e. of (A) and/or (B) may be, for example, an atomizable composition such as an aerosol comprising the active ingredient, i.e. (A) and (B) separately or in admixture, in solution or dispersion in a propellant, or a nebulizable composition comprising a solution or dispersion of the active ingredient in an aqueous, organic or aqueous/organic medium. For example, the inhalable form of the medicament may be an aerosol comprising a mixture of (A) and (B) in solution or dispersion in a propellant, or a combination of an aerosol containing (A) in solution or dispersion in a propellant with an aerosol containing (B) in solution or dispersion in a propellant. In another example, the inhalable form is a nebulizable composition comprising a dispersion of (A) and (B) in an aqueous, organic or aqueous/organic medium, or a combination of a dispersion of (A) in such a medium with a dispersion of (B) in such a medium.

An aerosol composition suitable for use as the inhalable form of the medicament may comprise the active ingredient in solution or dispersion in a propellant, which may be chosen from any of the propellants known in the art. Suitable such propellants include hydrocarbons such as n-propane, n-butane or isobutane or mixtures of two or more such hydrocarbons, and halogen-substituted hydrocarbons, for example chlorine and/or fluorine-substituted methanes, ethanes, propanes, butanes, cyclopropanes or cyclobutanes, such as dichlorodifluoromethane (CFC 12), trichlorofluoromethane (CFC11), 1,2-dichloro-1,1,2,2-tetrafluoroethane (CFC114) or, particularly, 1,1,1,2-tetrafluoroethane (HFA134a) and 1,1,1,2,3,3,3-heptafluoropropane (HFA227), or mixtures of two or more such halogen-substituted hydrocarbons. Where the active ingredient is present in suspension in the propellant, i.e. where it is present in particulate form dispersed in the propellant, the aerosol composition may also contain a lubricant and a surfactant, which may be chosen from those lubricants and surfactants known in the art. Other suitable aerosol compositions include surfactant-free or substantially surfactant-free aerosol compositions. The aerosol composition may contain up to about 5% by weight, for example 0.0001 to 5%, 0.001 to 5%, 0.001 to 3%, 0.001 to 2%, 0.001 to 1%, 0.001 to 0.1%, or 0.001 to 0.01% by weight of the active ingredient, based on the weight of the propellant. Where present, the lubricant and surfactant may be in an amount up to 5% and 0.5% respectively by weight of the aerosol composition. The aerosol composition may also contain a co-solvent such as ethanol in an amount up to 30% by weight of the composition, particularly for administration from a pressurised metered dose inhalation device. The aerosol composition may further contain a bulking agent, for example a sugar such as lactose, sucrose, dextrose, mannitol or sorbitol, in an amount, for example, of up to 20%, usually 0.001 to 1%, by weight of the composition.

In another embodiment of the invention, the inhalable form is a dry powder, i.e. (A) and/or (B) are present in a dry powder comprising finely divided (A) and/or (B) optionally together with at least one particulate pharmaceutically acceptable carrier, which may be one or more materials known as pharmaceutically acceptable carriers, preferably chosen from materials known as carriers in dry powder inhalation compositions, for example saccharides, including monosaccharides, disaccharides, polysaccharides and sugar alcohols such as arabinose, glucose, fructose, ribose, mannose, sucrose, trehalose, lactose, maltose, starches, dextran, mannitol or sorbitol. An especially preferred carrier is lactose. The dry powder may be contained as unit doses in capsules of, for example, gelatin or plastic, or in blisters (e.g. of aluminium or plastic), for use in a dry powder inhalation device, which may be a single dose or multiple dose device, preferably in dosage units of (A) and/or (B) together with the carrier in amounts to bring the total weight of powder per capsule to from 5 mg to 50 mg. Alternatively, the dry powder may be contained in a reservoir in a multi-dose dry powder inhalation device adapted to deliver, for example, 3-25 mg of dry powder per actuation.

In the finely divided particulate form of the medicament, and in the aerosol composition where the active ingredient is present in particulate form, the active ingredient may have an average particle diameter of up to about 10 μm, for example 0.1 to 5 μm, preferably 1 to 5 μm. The particulate carrier, where present, generally has a maximum particle diameter up to 300 μm, preferably up to 212 μm, and conveniently has a mean particle diameter of 40 to 100 μm, e.g. 50 to 75 μm. The particle size of the active ingredient, and that of a particulate carrier where present in dry powder compositions, can be reduced to the desired level by conventional methods, for example by grinding in an air-jet mill, ball mill or vibrator mill, sieving, microprecipitation, spray-drying, lyophilisation or controlled crystallisation from conventional solvents or from supercritical media.

The inhalable medicament may be administered using an inhalation device suitable for the inhalable form, such devices being well known in the art. Accordingly, the invention also provides a pharmaceutical product comprising a medicament or pharmaceutical composition as hereinbefore described in inhalable form as hereinbefore described in association with one or more inhalation devices. In a further aspect, the invention provides an inhalation device, or a pack of two or more inhalation devices, containing a medicament or pharmaceutical composition as hereinbefore described in inhalable form as hereinbefore described.

Where the inhalable form of the active ingredient is an aerosol composition, the inhalation device may be an aerosol vial provided with a valve adapted to deliver a metered dose, such as 10 to 100 μl, e.g. 25 to 50 μl, of the composition, i.e. a device known as a metered dose inhaler. Suitable such aerosol vials and procedures for containing within them aerosol compositions under pressure are well known to those skilled in the art of inhalation therapy. For example, an aerosol composition may be administered from a coated can, for example as described in EP-A-0642992. Where the inhalable form of the active ingredient is a nebulizable aqueous, organic or aqueous/organic dispersion, the inhalation device may be a known nebulizer, for example a conventional pneumatic nebulizer such as an airjet nebulizer, or an ultrasonic nebulizer, which may contain, for example, from 1 to 50 ml, commonly 1 to 10 ml, of the dispersion; or a hand-held nebulizer, sometimes referred to as a soft mist or soft spray inhaler, for example an electronically controlled device such as an AERx (Aradigm, US) or Aerodose (Aerogen), or a mechanical device such as a RESPIMAT (Boehringer Ingelheim) nebulizer which allows much smaller nebulized volumes, e.g. 10 to 100 μl, than conventional nebulizers. Where the inhalable form of the active ingredient is the finely divided particulate form, the inhalation device may be, for example, a dry powder inhalation device adapted to deliver dry powder from a capsule or blister containing a dry powder comprising a dosage unit of (A) and/or (B) or a multidose dry powder inhalation (MDPI) device adapted to deliver, for example, 3-25 mg of dry powder comprising a dosage unit of (A) and/or (B) per actuation. The dry powder composition preferably contains a diluent or carrier, such as lactose, and a compound that helps to protect against product performance deterioration due to moisture e.g. magnesium stearate, typically 0.05-1.5%. Suitable such dry powder inhalation devices are well known. For example, a suitable device for delivery of dry powder in encapsulated form is that described in U.S. Pat. No. 3,991,761, while a suitable MDPI device is that described in WO 97/20589.

The medicament of the invention is preferably a pharmaceutical composition comprising a mixture of (A) as hereinbefore defined and (B) as hereinbefore defined, preferably together with at least one pharmaceutically acceptable carrier as hereinbefore described.

The molar ratio of the compound (A) to the steroid (B) may be, in general, from 100:1 to 1:300, for example from 50:1 to 1:100 or from 20:1 to 1:50, preferably from 10:1 to 1:20, more preferably from 5:1 to 1:10, from 3:1 to 1:7 or from 2:1 to 1:2. The compound (A) and the steroid (B) may be administered separately in the same ratio.

A suitable daily dose of the compound (A), particularly as the maleate salt, for inhalation may be from 20 μg to 2000 μg, for example from 20 to 1500 μg, from 20 to 1000 μg, preferably from 50 to 800 μg, e.g. from 100 to 600 μg or from 100 to 500 μg.

Where (B) is a PDE4 inhibitor, a suitable daily dose for inhalation may be from 20 μg to 5000 μg, for example from 20 to 4000 μg, from 50 to 3000 μg, from 50 to 2000 μg, from 50 to 1000 μg, from 50 to 500 μg, from 50 to 400 μg, from 50 to 300 μg, from 50 to 200 μg or from 50 to 100 μg.

Where (B) is a PDE5 inhibitor, a suitable daily dose for inhalation may be from 20 μg to 5000 μg, for example from 20 to 4000 μg, from 50 to 3000 μg, from 50 to 2000 μg, from 50 to 1000 μg, from 50 to 500 μg, from 50 to 400 μg, from 50 to 300 μg, from 50 to 200 μg or from 50 to 100 μg.

In one preferred embodiment of the invention, the medicament of the invention is a pharmaceutical composition which is a dry powder in a capsule containing a unit dose of (A) and (B), for example for inhalation from a single capsule inhaler, the capsule suitably containing a unit dose of (A) e.g. as hereinbefore described, and a unit dose of (B), e.g. as hereinbefore described, together with a pharmaceutically acceptable carrier as hereinbefore described in an amount to bring the total weight of dry powder per capsule to between 5 mg and 50 mg, for example 5 mg, 10 mg, 15 mg, 20 mg, 25 mg, 30 mg, 35 mg, 40 mg, 45 mg or 50 mg.

In another preferred embodiment of the invention, the medicament of the invention is a pharmaceutical composition which is a dry powder for administration from a reservoir of a multi-dose dry powder inhaler adapted to deliver, for example, 3 mg to 25 mg of powder containing a unit dose of (A) and (B) per actuation, for example, where (A) is in the form of the maleate salt, a powder comprising, by weight, 20 to 2000 parts, for example 60 to 1000 parts, 100 to 500 parts, or 100 to 300 parts of (A); 25 to 800 parts, e.g. 25 to 500 parts, 50 to 400 parts, or 100 to 400 parts of (B); and 2000 to 25000 parts, e.g. 4000 to 15000 parts or 4000 to 10000 parts of a pharmaceutically acceptable carrier as hereinbefore described.

In a further preferred embodiment of the invention, the medicament of the invention is a pharmaceutical composition which is an aerosol comprising (A) and (B), e.g. in a ratio as hereinbefore described, in a propellant as hereinbefore described, optionally together with a surfactant and/or a bulking agent and/or a co-solvent such as ethanol as hereinbefore described, for administration from a metered dose inhaler adapted to deliver an amount of aerosol containing a unit dose of (A) and a unit dose of (B), or a known fraction of a unit dose of (A) and a known fraction of a unit dose of (B), per actuation. Thus if, for example, the inhaler delivers half of the unit doses of (A) and (B) per actuation, the unit doses can be administered by two actuations of the inhaler.

In accordance with the above, the invention also provides a pharmaceutical kit comprising (A) and (B) as hereinbefore defined in separate unit dosage forms, said forms being suitable for administration of (A) and (B) in effective amounts. Such a kit suitably further comprises one or more inhalation devices for administration of (A) and (B). For example, the kit may comprise one or more dry powder inhalation devices adapted to deliver dry powder from a capsule, together with capsules containing a dry powder comprising a dosage unit of (A) and capsules containing a dry powder comprising a dosage unit of (B). In another example, the kit may comprise a multidose dry powder inhalation device containing in the reservoir thereof a dry powder comprising (A) and a multidose dry powder inhalation device containing in the reservoir thereof a dry powder comprising (B). In a further example, the kit may comprise a metered dose inhaler containing an aerosol comprising (A) in a propellant and a metered dose inhaler containing an aerosol comprising (B) in a propellant.

The medicaments of the invention are advantageous in the treatment of inflammatory or obstructive airways disease, exhibiting highly effective bronchodilatory and anti-inflammatory properties. For instance, it is possible using the combination therapy of the invention to reduce the dosages of corticosteroid required for a given therapeutic effect compared with those required using treatment with a corticosteroid alone, thereby minimising possibly undesirable side effects. In particular, these combinations, particularly where (A) and (B) are in the same composition, facilitate achievement of a high anti-inflammatory effect, such that the amount of corticosteroid needed for a given anti-inflammatory effect may be reduced when used in admixture with a compound of formula I, thereby reducing the risk of undesirable side effects from the repeated exposure to the steroid involved in the treatment of inflammatory or obstructive airways diseases. Furthermore, using the combinations of the invention, particularly using compositions containing (A) and (B), medicaments which have a rapid onset of action and a long duration of action may be prepared. Moreover, using such combination therapy, medicaments which result in a significant improvement in lung function may be prepared. In another aspect, using the combination therapy of the invention, medicaments which provide effective control of obstructive or inflammatory airways diseases, or a reduction in exacerbations of such diseases, may be prepared. In a further aspect, using compositions of the invention containing (A) and (B), medicaments which reduce or eliminate the need for treatment with short-acting rescue medicaments such as salbutamol or terbutaline, may be prepared; thus compositions of the invention containing (A) and (B) facilitate the treatment of an obstructive or inflammatory airways disease with a single medicament.

Treatment of inflammatory or obstructive airways diseases in accordance with the invention may be symptomatic or prophylactic treatment. Inflammatory or obstructive airways diseases to which the present invention is applicable include asthma of whatever type or genesis including both intrinsic (non-allergic) asthma and extrinsic (allergic) asthma, mild asthma, moderate asthma, severe asthma, bronchitic asthma, exercise-induced asthma, occupational asthma and asthma induced following bacterial infection. Treatment of asthma is also to be understood as embracing treatment of subjects, e.g. of less than 4 or 5 years of age, exhibiting wheezing symptoms and diagnosed or diagnosable as “wheezy infants”, an established patient category of major medical concern and now often identified as incipient or early-phase asthmatics. (For convenience this particular asthmatic condition is referred to as “wheezy-infant syndrome”.)

Prophylactic efficacy in the treatment of asthma will be evidenced by reduced frequency or severity of symptomatic attack, e.g. of acute asthmatic or bronchoconstrictor attack, improvement in lung function or improved airways hyperreactivity. It may further be evidenced by reduced requirement for other, symptomatic therapy, i.e. therapy for or intended to restrict or abort symptomatic attack when it occurs, for example anti-inflammatory (e.g. corticosteroid) or bronchodilatory. Prophylactic benefit in asthma may in particular be apparent in subjects prone to “morning dipping”. “Morning dipping” is a recognised asthmatic syndrome, common to a substantial percentage of asthmatics and characterised by asthma attack, e.g. between the hours of about 4 to 6 am, i.e. at a time normally substantially distant form any previously administered symptomatic asthma therapy.

Other inflammatory or obstructive airways diseases and conditions to which the present invention is applicable include acute lung injury (ALI), adult respiratory distress syndrome (ARDS), chronic obstructive pulmonary, airways or lung disease (COPD, COAD or COLD), including chronic bronchitis and emphysema, bronchiectasis and exacerbation of airways hyperreactivity consequent to other drug therapy, in particular other inhaled drug therapy. Further inflammatory or obstructive airways diseases to which the present invention is applicable include pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tobacosis and byssinosis.

The invention is illustrated by the following Examples.

### EXAMPLES

### Compound A1

### (R)-5-[2-(5,6-diethyl-indan-2-ylamino)-1-hydroxy-ethyl]-8-hydroxy-1H-quinolin-2-one maleate

This compound is prepared using the procedures described in international patent application WO 2000/075114.

### Compound B1

This compound is the PDE4 inhibitor cilomilast.

### Compound B2

This compound is the PDE4 inhibitor roflumilast.

### Compound B3

This compound is the PDE5 inhibitor sildenafil.

### Compound B4

This compound is the PDE5 inhibitor tadafil.

### Compound B5

This compound is the PDE5 inhibitor vardenafil.

### Examples 1-60

Gelatin capsules suitable for use in a capsule inhaler such as that described in U.S. Pat. No. 3,991,761 and EP 1270034 are prepared, each capsule containing a dry powder obtained by mixing Compound A1 and Compound B1 which have been ground to a mean particle diameter of 1 to 5 μm and lactose monohydrate having a particle diameter below 212 μm, the amounts being as shown in the Table 1 below:

TABLE 1

| | Compound A1 | Compound B1 | Lactose |
|---|---|---|---|
| Example | (Parts) | (Parts) | (Parts) |
| 1 | 20 | 100 | 19880 |
| 2 | 40 | 100 | 19860 |
| 3 | 80 | 100 | 19820 |
| 4 | 100 | 100 | 19800 |
| 5 | 120 | 100 | 19780 |
| 6 | 140 | 100 | 19760 |
| 7 | 160 | 100 | 19740 |
| 8 | 180 | 100 | 19720 |
| 9 | 200 | 100 | 19700 |
| 10 | 220 | 100 | 19680 |
| 11 | 240 | 100 | 19660 |
| 12 | 300 | 100 | 19600 |
| 13 | 500 | 100 | 19400 |
| 14 | 1000 | 100 | 18900 |
| 15 | 2000 | 100 | 17900 |
| 16 | 20 | 100 | 24880 |
| 17 | 40 | 100 | 24860 |
| 18 | 80 | 100 | 24820 |
| 19 | 100 | 100 | 24800 |
| 20 | 120 | 100 | 24780 |
| 21 | 140 | 100 | 24760 |
| 22 | 160 | 100 | 24740 |
| 23 | 180 | 100 | 24720 |
| 24 | 200 | 100 | 24700 |
| 25 | 220 | 100 | 24680 |
| 26 | 240 | 100 | 24660 |
| 27 | 300 | 100 | 24600 |
| 28 | 500 | 100 | 24400 |
| 29 | 1000 | 100 | 23900 |
| 30 | 2000 | 100 | 22900 |
| 31 | 20 | 200 | 14780 |
| 32 | 40 | 200 | 14760 |
| 33 | 80 | 200 | 14720 |
| 34 | 100 | 200 | 14700 |
| 35 | 120 | 200 | 14680 |
| 36 | 140 | 200 | 14660 |
| 37 | 160 | 200 | 14640 |
| 38 | 180 | 200 | 14620 |
| 39 | 200 | 200 | 14600 |
| 40 | 220 | 200 | 14580 |
| 41 | 240 | 200 | 14560 |
| 42 | 300 | 200 | 14500 |
| 43 | 500 | 200 | 14300 |
| 44 | 1000 | 200 | 13800 |
| 45 | 2000 | 200 | 12800 |
| 46 | 20 | 200 | 24780 |
| 47 | 40 | 200 | 24760 |
| 48 | 80 | 200 | 24720 |
| 49 | 100 | 200 | 24700 |
| 50 | 120 | 200 | 24680 |
| 51 | 140 | 200 | 24660 |
| 52 | 160 | 200 | 24640 |
| 53 | 180 | 200 | 24620 |
| 54 | 200 | 200 | 24600 |
| 55 | 220 | 200 | 24580 |
| 56 | 240 | 200 | 24560 |
| 57 | 300 | 200 | 24500 |
| 58 | 500 | 200 | 24300 |
| 59 | 1000 | 200 | 23800 |
| 60 | 2000 | 200 | 22800 |

### Examples 61-105

A dry powder suitable for delivery from the reservoir of the multi-dose inhaler described in WO 97/20589 is prepared by mixing Compound A1 and Compound B2 which have been ground to a mean particle diameter of 1-5 μm and lactose monohydrate having a particle diameter below 212 μm, the amounts being as shown in the Table 2 below:

TABLE 2

| | Compound A1 | Compound B2 | Lactose |
|---|---|---|---|
| Example | (Parts) | (Parts) | (Parts) |
| 61 | 20 | 100 | 4880 |
| 62 | 40 | 100 | 4860 |
| 63 | 80 | 100 | 4820 |
| 64 | 100 | 100 | 4800 |
| 65 | 120 | 100 | 4780 |
| 66 | 140 | 100 | 4760 |
| 67 | 160 | 100 | 4740 |
| 68 | 180 | 100 | 4720 |
| 69 | 200 | 100 | 4700 |
| 70 | 220 | 100 | 4680 |
| 71 | 240 | 100 | 4660 |
| 72 | 300 | 100 | 4600 |
| 73 | 500 | 100 | 4400 |
| 74 | 1000 | 100 | 3900 |
| 75 | 2000 | 100 | 2900 |
| 76 | 20 | 200 | 9780 |
| 77 | 40 | 200 | 9760 |
| 78 | 80 | 200 | 9720 |
| 79 | 100 | 200 | 9700 |
| 80 | 120 | 200 | 9680 |
| 81 | 140 | 200 | 9660 |
| 82 | 160 | 200 | 9640 |
| 83 | 180 | 200 | 9620 |
| 84 | 200 | 200 | 9600 |
| 85 | 220 | 200 | 9580 |
| 86 | 240 | 200 | 9560 |
| 87 | 300 | 200 | 9500 |
| 88 | 500 | 200 | 9300 |
| 89 | 1000 | 200 | 8800 |
| 90 | 2000 | 200 | 7800 |
| 91 | 20 | 250 | 14730 |
| 92 | 40 | 250 | 14710 |
| 93 | 80 | 250 | 14670 |
| 94 | 100 | 250 | 14650 |
| 95 | 120 | 250 | 14630 |
| 96 | 140 | 250 | 14610 |
| 97 | 160 | 250 | 14590 |
| 98 | 180 | 250 | 14570 |
| 99 | 200 | 250 | 14550 |
| 100 | 220 | 250 | 14530 |
| 101 | 240 | 250 | 14510 |
| 102 | 300 | 250 | 14450 |
| 103 | 500 | 250 | 14250 |
| 104 | 1000 | 250 | 13750 |
| 105 | 2000 | 250 | 12750 |

### Examples 106-150

A dry powder suitable for delivery from the reservoir of the multi-dose inhaler described in W0 97/20589 is prepared by mixing Compound A1 and Compound B3 which have been ground to a mean particle diameter of 1-5 μm and lactose monohydrate having a particle diameter below 212 μm, the amounts being as shown in the Table 2 but also containing 0.5% magnesium stearate by weight.

### Examples 151-168

Aerosol formulations are prepared by dispensing micronised active ingredients, Compound A1 and Compound B4, and if required, lactose as bulking agent into a vial, sealing the vial with a metering valve, injecting the premixed ethanol/propellant and optional surfactant into the vial through the valve and subjecting the vial to ultrasonic energy to disperse the solid particles. The components and amounts used are shown in Table 3 below:

TABLE 3

| | Cpd. A1 | Cpd. B4 | HFA134a | HFA227 | Ethanol | OA | Lactose |
|---|---|---|---|---|---|---|---|
| Ex. | (Parts) | (Parts) | (Parts) | (Parts) | (Parts) | (Parts) | (Parts) |
| 151 | 2 | 10 | 36500 | 60750 | 2500 | — | 70 |
| 152 | 4 | 10 | 3410 | 6340 | 230 | 0.3 | — |
| 153 | 8 | 10 | 97000 | — | 2500 | — | 90 |
| 154 | 10 | 10 | 30500 | 67000 | 2500 | 0.5 | 100 |
| 155 | 12 | 10 | 3150 | 6550 | 250 | 1 | — |
| 156 | 14 | 10 | 3700 | 6050 | 250 | 0.8 | — |
| 157 | 16 | 10 | 3800 | 5900 | 230 | 0.4 | — |
| 158 | 18 | 10 | 4700 | 5050 | 250 | 1 | — |
| 159 | 20 | 20 | 3600 | 6150 | 225 | 1 | — |
| 160 | 22 | 20 | 3500 | 6200 | 230 | 1 | — |
| 161 | 24 | 20 | 98000 | — | 2500 | 1 | — |
| 162 | 30 | 20 | 3900 | 5900 | 250 | 1 | — |
| 163 | 2 | 20 | 30000 | 67000 | 2250 | 0.2 | 90 |
| 164 | 10 | 20 | 3500 | 6200 | 250 | 0.5 | — |
| 165 | 14 | 20 | 3200 | 6500 | 230 | 1 | — |
| 166 | 18 | 20 | 3100 | 6200 | 225 | 0.8 | — |
| 167 | 20 | 20 | 3150 | 6100 | 225 | 1 | — |
| 168 | 24 | 20 | 30000 | 60000 | 2000 | 0.8 | — |

### Examples 169-178

Aerosol formulations are prepared by dispensing micronised active ingredients, Compound A1 and Compound B5, and if required, lactose as bulking agent into a vial, sealing the vial with a metering valve, injecting the premixed ethanol/propellant and optional surfactant into the vial through the valve and subjecting the vial to ultrasonic energy to disperse the solid particles. The components and amounts used are shown in Table 4 below:

TABLE 4

| | Cpd. A1 | Cpd. B5 | HFA134a | HFA227 | Ethanol | OA | Lactose |
|---|---|---|---|---|---|---|---|
| Ex. | (Parts) | (Parts) | (Parts) | (Parts) | (Parts) | (Parts) | (Parts) |
| 169 | 4 | 10 | 34000 | 63000 | 2250 | 0.3 | 50 |
| 170 | 8 | 10 | 92000 | — | 2500 | 0.5 | 70 |
| 171 | 12 | 10 | 3000 | 5500 | 200 | — | — |
| 172 | 16 | 10 | 2500 | 5000 | 200 | 0.3 | — |
| 173 | 20 | 10 | 2000 | 3000 | 150 | 0.2 | — |
| 174 | 30 | 10 | 2000 | 2000 | 150 | 0.2 | — |
| 175 | 8 | 20 | 20000 | 25000 | 1500 | 0.2 | — |
| 176 | 12 | 20 | 2500 | 2500 | 200 | 0.2 | — |
| 177 | 20 | 20 | 2000 | 2000 | 150 | 0.2 | — |
| 178 | 30 | 20 | 20000 | 20000 | 1500 | 0.2 | — |

1. A medicament comprising, separately or together,
(A) a compound of formula I 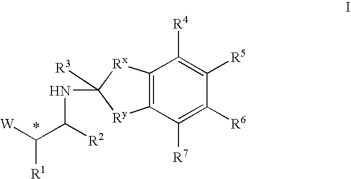
in free or salt or solvate form, where
W is a group of formula 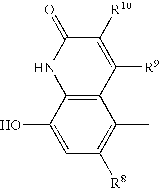
R^{x} and R^{y} are both —CH₂— or —(CH₂)₂—;
R¹ is hydrogen, hydroxy, or C₁-C₁₀-alkoxy;
R² and R³ are each independently hydrogen or C₁-C₁₀-alkyl;
R⁴, R⁵, R⁶ and R⁷ are each independently hydrogen, halogen, cyano, hydroxy, C₁-C₁₀-alkoxy, C₆-C₁₀-aryl, C₁-C₁₀-alkyl, C₁-C₁₀-alkyl substituted by one or more halogen atoms or one or more hydroxy or C₁-C₁₀-alkoxy groups, C₁-C₁₀-alkyl interrupted by one or more hetero atoms, C₂-C₁₀-alkenyl, trialkylsilyl, carboxy, C₁-C₁₀-alkoxycarbonyl, or —CONR¹¹R¹² where R¹¹ and R¹² are each independently hydrogen or C₁-C₁₀-alkyl,
or R⁴ and R⁵, R⁵ and R⁶, or R⁶ and R⁷ together with the carbon atoms to which they are attached denote a 5-, 6- or 7-membered carbocyclic ring or a 4- to 10-membered heterocyclic ring; and
R⁸, R⁹ and R¹⁰ are each independently hydrogen or C₁-C₄-alkyl; and
(B) one or more of compounds selected from the group consisting of PDE4 inhibitors and PDE5 inhibitors;
for simultaneous, sequential or separate administration in the treatment of an inflammatory or obstructive airways disease.

2. A medicament according to claim 1 which is a pharmaceutical composition comprising a mixture of effective amounts of (A) and (B) optionally together with at least one pharmaceutically acceptable carrier.

3. A medicament according to claim 1, in which (A) is a compound of formula I wherein
R⁸, R⁹ and R¹⁰ are each H, R¹ is OH, R² and R³ are each H and
(i) R^{x} and R^{y} are both —CH₂—, and R⁴ and R⁷ are each CH₃O— and R⁵ and R⁶ are each H;
(ii) R^{x} and R^{y} are both —CH₂—, and R⁴ and R⁷ are each H and R⁵ and R⁶ are each CH₃CH₂—;
(iii) R^{x} and R^{y} are both —CH₂—, and R⁴ and R⁷ are each H and R⁵ and R⁶ are each CH₃—;
(iv) R^{x} and R^{y} are both —CH₂—, and R⁴ and R⁷ are each CH₃CH₂— and R⁵ and R⁶ are each H;
(v) R^{x} and R^{y} are both —CH₂—, and R⁴ and R⁷ are each H and R⁵ and R⁶ together denote —(CH₂)₄—;
(vi) R^{x} and R^{y} are both —CH₂—, and R⁴ and R⁷ are each H and R⁵ and R⁶ together denote —O(CH₂)₂O—;
(vii) R^{x} and R^{y} are both —CH₂—, and R⁴ and R⁷ are each H and R⁵ and R⁶ are each CH₃(CH₂)₃—;
(viii) R^{x} and R^{y} are both —CH₂—, and R⁴ and R⁷ are each H and R⁵ and R⁶ are each CH₃(CH₂)₂—;
(ix) R^{x} and R^{y} are both —(CH₂)₂—, R⁴, R⁵, R⁶ and R⁷ are each H; or
(x) R^{x} and R^{y} are both —CH₂—, and R⁴ and R⁷ are each H and R⁵ and R⁶ are each CH₃OCH₂—.

4. A medicament according to claim 1, in which (A) is a compound of formula II 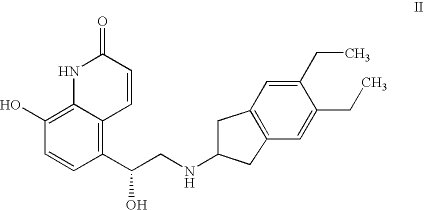
in free or pharmaceutically acceptable salt or solvate form.

5. A medicament according to claim 4, in which (A) is the maleate salt.

6. A medicament according to claim 1, in which (B) is a PDE4 inhibitor selected from the group consisting of cilomilast, roflumilast, V-11294A, BAY19-8004, SCH-351591, arofylline, PD189659, PD168787, AWD-12-281, CDC-801, CC-10004, VM554/UM565, T-440 and KW-4490.

7. A medicament according to claim 1, in which (B) is a PDE5 inhibitor selected from the group consisting of 5-[2-ethoxy-5-(4-methylpiperazinylsulfonyl)-phenyl]-1-methyl-3-n-propyl-1,6-dihydro-7H-pyrazolo[4,3-d]pyrimidin-7-one, (6R,12aR)-2,3,6,7,12,12a-Hexahydro-2-methyl-6-(3,4-methylenedioxyphenyl)-pyrazino(2′,1′:6,1)pyrido (3,4-b)indole-1,4-dione, 4-ethoxy-N-propyl-N-(2-hydroxyethyl)-3-(5-methyl-4-oxo-7-propyl-3,4-dihydroimidazo[5,1-f][1,2,4]triazin-2-yl)benzenesulfonamide, 4-phenyl-methylamino-6-chloro-2-(1-imidazolyl)-quinazoline, 4-phenylmethylamino-6-chloro-2-(3-pyridyl)quinazoline, 1,3-dimethyl-6-(2-propoxy-5-methanesulfonylamidophenyl)-1,5-dihydropyrazolo[3,4-d]pyrimidin-4-one and 1-cyclopentyl-3-ethyl-6-(3-ethoxy-4-pyridyl)-pyrazolo[3,4-d]pyrimidin-4-one.

8. A medicament according to claim 1 in inhalable form as an aerosol comprising a mixture of (A) and (B) in solution or dispersion in a propellant, or a combination of an aerosol containing (A) in solution or dispersion in a propellant with an aerosol containing (B) in solution or dispersion in a propellant.

9. A medicament according to claim 1 in the inhalable form as a nebulizable composition comprising a dispersion of (A) and (B) in an aqueous, organic or aqueous/organic medium or a combination of a dispersion of (A) in said medium with a dispersion of (B) in said medium.

10. A medicament according to claim 1 in which (A) and/or (B) are present in inhalable form as a dry powder comprising finely divided (A) and/or (B) optionally together with at least one particulate pharmaceutically acceptable carrier.

11. A medicament according to claim 8, in which (A) and/or (B) has an average particle diameter up to 10 μm.

12. A medicament according to claim 1, in which the molar ratio of (A) to (B) is from 5:1 to 1:10.

13. A medicament according to claim 2, which is a dry powder in a capsule, the capsule containing a unit dose of (A), a unit dose of (B) and a pharmaceutically acceptable carrier in an amount to bring the total weight of dry powder per capsule to between 5 mg and 50 mg.

14. A medicament according to claim 2, which is a dry powder comprising, by weight, from 20 to 2000 parts of (A) in the form of the maleate salt, from 25 to 800 parts of (B) and 2000 to 25000 parts of a pharmaceutically acceptable carrier.

15. A medicament according to claim 2, which is an aerosol comprising (A) and (B) in a ratio as hereinbefore specified in claim 1 or 12, in a propellant, optionally together with a surfactant and/or a bulking agent and/or a co-solvent suitable for administration from a metered dose inhaler adapted to deliver an amount of aerosol containing a unit dose of (A) and a unit dose of (B), or a known fraction of a unit dose of (A) and a known fraction of a unit dose of (B), per actuation.

16. A pharmaceutical kit comprising (A) as defined in claim 1 and (B) as defined in claim 1 in separate unit dosage forms, said forms being suitable for administration of (A) and (B) in effective amounts, together with one or more inhalation devices for administration of (A) and (B).

17. A medicament substantially as herein described with reference to any one of the Examples.
